(19) United States
(12) Patent Application Publication
(10) Pub. No.: US 2016/0120529 A1
(43) Pub. Date: May 5, 2016

(54) SURGICAL INSTRUMENT AND METHOD

(71) Applicant: Warsaw Orthopedic, Inc., Warsaw, IN (US)

(72) Inventors: Cristian A. Capote, Memphis, TN (US); Michelle M. Hennard, Memphis, TN (US)

(21) Appl. No.: 14/529,951

(22) Filed: Oct. 31, 2014

(51) Int. Cl. A61B 17/02; A61B 17/70

(52) U.S. Cl. CPC A61B 17/025; A61B 17/7074; A61B 2017/0256; A61B 2019/4857

(57) ABSTRACT

A surgical instrument includes a first arm that defines a longitudinal axis and includes at least one tissue engaging member being movable along a transverse axis. A second arm is translatable relative to the first arm and includes at least one tissue engaging member being movable along a transverse axis thereof. The tissue engaging members are relatively movable between a first configuration and a second configuration to space tissue. Systems and methods are disclosed.

### TECHNICAL FIELD

The present disclosure generally relates to medical devices for the treatment of musculoskeletal disorders, and more particularly to a surgical system and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods, connectors, plates and interbody devices can be used to provide stability to a treated region. For example, during surgical treatment, surgical instruments can be used to deliver components of the spinal constructs to the surgical site for fixation with bone to immobilize a joint. Surgical instruments, such as, for example, retractors may be employed during a surgical treatment to provide access and visualization of a surgical site. Such retractors space apart and support tissue and/or other anatomical structures to expose anatomical structures adjacent the surgical site and/or provide a surgical pathway to the surgical site. This disclosure describes an improvement over these prior art technologies.

### SUMMARY

In one embodiment, a surgical instrument is provided. The surgical instrument comprises a first arm that defines a longitudinal axis and includes at least one tissue engaging member being movable along a transverse axis. A second arm is translatable relative to the first arm and includes at least one tissue engaging member being movable along a transverse axis thereof. The tissue engaging members are relatively movable between a first configuration and a second configuration to space tissue. In some embodiments, systems and methods are disclosed.

### BRIEF DESCRIPTION OF THE DRAWINGS

The present disclosure will become more readily apparent from the specific description accompanied by the following drawings, in which:

FIG. 1 is a plan view of one embodiment of components of a system in accordance with the principles of the present disclosure;

FIG. 2 is a plan view of components of the system shown in FIG. 1;

FIG. 3 is a side view of the components shown in FIG. 1;

FIG. 4 is a side view of the components shown in FIG. 1; and

FIG. 5 is a plan view of one embodiment of components of a system in accordance with the principles of the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical system for delivery to a surgical site and a method for treating a spine.

In one embodiment, the surgical system includes a surgical instrument, such as, for example, a retractor. In some embodiments, the system includes a retractor configured to provide guidance for screw orientation. In one embodiment, the system includes a retractor configured to facilitate tissue retraction and provide visual guidance to facilitate screw placement and trajectory. In one embodiment, the retractor includes four retraction blades attached to a mechanism to allow independent positioning of each of the four retractor blades. In some embodiments, the system includes blades having fluoroscopic or other visual indicators to facilitate screw trajectory and placement to provide a visual indication to facilitate alignment of the instrument during screw insertion to achieve a desired screw trajectory. In one embodiment, the system includes a visual indicator that indicates a screw starting point. In some embodiments, the system includes a retractor that provides a mechanical guidance mechanism to maintain trajectory. In some embodiments, the system includes retractor blades positioned in alignment with a screw trajectory such that retraction only occurs in a specific area such that tissue is not over or unnecessarily retracted.

In one embodiment, the system includes retractor blades configured for inside out dilation such that the retractor blades open from a closed position. In some embodiments, this configuration allows for positioning of the retractor blades along a screw trajectory to achieve minimal soft tissue disruption, establish a visual cue for screw guidance and limit the retraction to a surgical site.

In one embodiment, the system includes a first pair of blades and a second pair of blades. In one embodiment, the first pair of blades is configured for lateral translation. In one embodiment, the second pair of blades is configured for lateral translation independent of the first pair of blades. In some embodiments, the first pair of blades is configured for cranial and/or caudal translation. In some embodiments, the second pair of blades is configured for cranial and/or caudal translation.

In one embodiment, the system includes a mechanism to facilitate independent rotation of the blades. In one embodiment, the blades are aligned only in a specific area being prepared for screw placement. In one embodiment, the system utilized fluoroscopic alignment such that an axis of the blades is aligned with an axis of the screw trajectory. In some embodiments, the system includes guidance for screw docking points. In some embodiments, the system includes visual feedback of the screw trajectory.

In one embodiment, the present disclosure may be employed to treat spinal disorders, such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

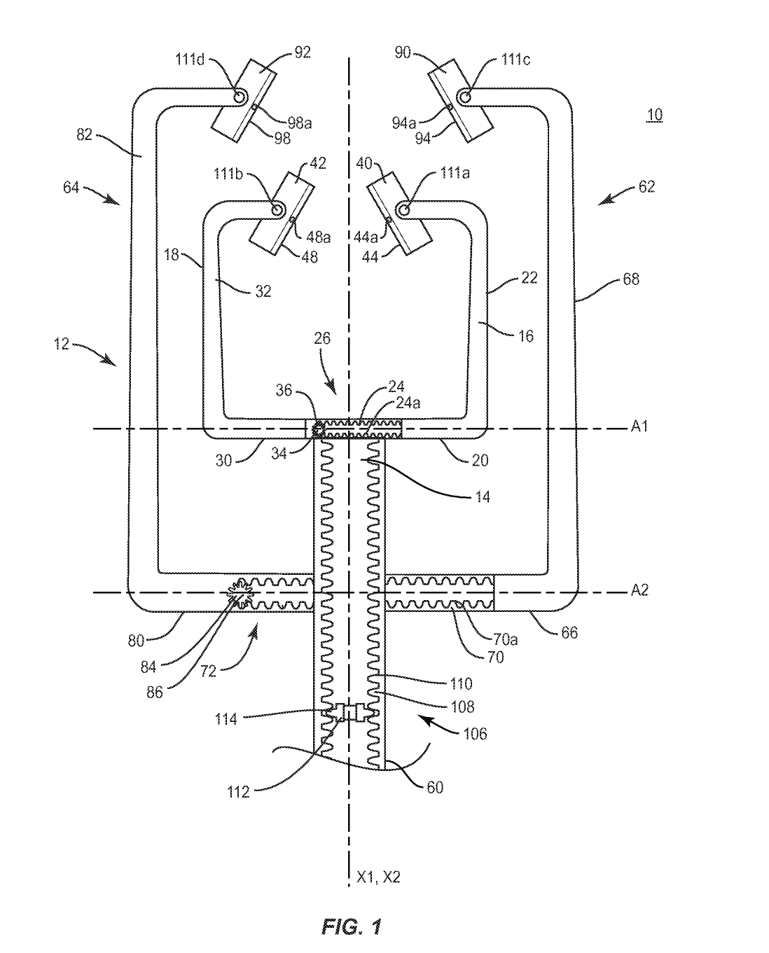

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-4, there are illustrated components of a surgical system 10.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, super elastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of surgical system 10 may have material composites, including the above materials, to achieve various desired characteristics such as strength, rigidity, elasticity, compliance, biomechanical performance, durability and radiolucency or imaging preference. The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

Surgical system 10 is employed, for example, with a fully open surgical procedure, a minimally invasive procedure, including percutaneous techniques, and mini-open surgical techniques to deliver and introduce instrumentation and/or an implant, such as, for example, an interbody implant, at a surgical site within a subject body of a patient, which includes, for example, a spine having vertebrae V (FIG. 5). In some embodiments, the implant can include spinal constructs, such as, for example, interbody devices, cages, bone fasteners, spinal rods, connectors and/or plates.

Surgical system 10 includes a surgical instrument, such as, for example, a retractor 12. Retractor 12 includes an arm 14 that defines a longitudinal axis X1. Arm 14 is translatable relative to an arm 60, which defines an axis X2, as described herein. In one embodiment, arms 14, 60 are coaxially disposed and configured for independent and/or relative translation along axes X1, X2. In some embodiments, arms 14, 60 may be disposed in various relative orientations, such as, for example, series, parallel, offset, staggered.

Arm 14 includes a member 16 and a member 18. Members 16, 18 are configured for independent and/or relative translation, as described herein. In some embodiments, members 16, 18 are configured for independent and/or relative rotation. Member 16 includes a member 20, which is disposed in a transverse orientation relative to axis X1 and a member 22, which is disposed in an offset and/or parallel orientation relative to axis X1. Member 22 extends from member 20 in a perpendicular orientation. In some embodiments, member 22 may be disposed in transverse orientations relative to member 20, such as, for example, angular orientations such as acute or obtuse, and/or may be offset or staggered. In one embodiment, member 16 includes an engagement portion, such as, example, a carriage comprising a rack 24 of a ratchet mechanism 26 for incremental and controlled independent and/or relative translation of members 16, 18 to facilitate movement of blade 40 and/or blade 42, which may include independent and/or relative translation of blade 40 and/or blade 42, as described herein. Rack 24 includes gear teeth 24a disposed in a linear configuration along member 20 for engagement with member 18 to facilitate incremental and controlled independent and/or relative translation of members 16, 18 along a transverse axis A1, as described herein.

Member 18 includes a member 30, which is disposed in a transverse orientation relative to axis X1 and a member 32, which is disposed in an offset and/or parallel orientation relative to axis X1. Member 32 extends from member 30 in a perpendicular orientation. In some embodiments, member 32 may be disposed in transverse orientations relative to member 30, such as, for example, angular orientations such as acute or obtuse, and/or may be offset or staggered. Members 20, 30 are engageable to facilitate incremental and controlled independent and/or relative translation of members 16, 18 along axis A1. In one embodiment, member 30 includes an engagement portion, such as, example, pawls 34. Pawls 34 include gear teeth 36. Gear teeth 36 are configured for engagement with gear teeth 24a of rack 24 to facilitate incremental and controlled independent and/or relative translation of members 16, 18 along axis A1 to facilitate movement of blade 40 and/or blade 42, as described herein. In some embodiments, members 20, 30 include a rack and rotatable gear, such as, for example, a rack and pinion gear configuration.

Member 16 includes a tissue engaging member, such as, for example, a blade 40 and member 18 includes a tissue engaging member, such as, for example, a blade 42. Blade 40 extends from member 22 and blade 42 extends from member 32. Blade 40 extends between an end 40a and an end 40b. Blade 40 includes an inner surface, such as, for example, a guide surface 44 and an outer surface 46 configured for engagement with tissue. Blade 42 extends between an end 42a and an end 42b. Blade 42 includes an inner surface, such as, for example, a guide surface 48 and an outer surface 50 configured for engagement with tissue. In some embodiments, all or only a portion of blade 40 and/or blade 42 may have various cross-section configurations, such as, for example, arcuate, cylindrical, oblong, rectangular, polygonal, undulating, irregular, uniform, non-uniform, consistent, variable, and/or U-shape.

Blades 40, 42 are independently, and/or relatively translatable and/or rotatable along axis X1 and axis A1. Blades 40, 42 are connected with members 16, 18 for movement, as described herein, to facilitate manipulation of tissue in and between one or a plurality of configurations and to space the tissue and define a configuration and dimension of a surgical opening, such as, for example, a surgical pathway, as described herein. In some embodiments, blade 40 and/or blade 42 includes an adjustable length such that a first portion of blade 40 and/or blade 42 translates relative to a second portion of blade 40 and/or blade 42.

Arm 60 includes a member 62 and a member 64. Members 62, 64 are configured for independent and/or relative translation, as described herein. In some embodiments, members 62, 64 are configured for independent and/or relative rotation. Member 62 includes a member 66, which is disposed in a transverse orientation relative to axis X2 and a member 68, which is disposed in an offset and/or parallel orientation relative to axis X2. Member 68 extends from member 66 in a perpendicular orientation. In some embodiments, member 68 may be disposed in transverse orientations relative to member 66, such as, for example, angular orientations such as acute or obtuse, and/or may be offset or staggered. In one embodiment, member 62 includes an engagement portion, such as, example, a carriage comprising a rack 70 of a ratchet mechanism 72 for incremental and controlled independent and/or relative translation of members 62, 64 to facilitate movement of blade 90 and/or blade 92, which may include independent and/or relative translation of blade 90 and/or blade 92, as described herein. Rack 70 includes gear teeth 70a disposed in a linear configuration along member 66 for engagement with member 64 to facilitate incremental and controlled independent and/or relative translation of members 62, 64 along a transverse axis A2, as described herein.

Member 64 includes a member 80, which is disposed in a transverse orientation relative to axis X2 and a member 82, which is disposed in an offset and/or parallel orientation relative to axis X2. Member 82 extends from member 80 in a perpendicular orientation. In some embodiments, member 82 may be disposed in transverse orientations relative to member 80, such as, for example, angular orientations such as acute or obtuse, and/or may be offset or staggered. Members 66, 80 are engageable to facilitate incremental and controlled independent and/or relative translation of members 62, 64 along axis A2. In one embodiment, member 80 includes an engagement portion, such as, example, pawls 84. Pawls 84 include gear teeth 86. Gear teeth 86 are configured for engagement with gear teeth 70a of rack 70 to facilitate incremental and controlled independent and/or relative translation of members 62, 64 along axis A2 to facilitate movement of blade 90 and/or blade 92, as described herein. In some embodiments, members 66, 80 include a rack and rotatable gear, such as, for example, a rack and pinion gear configuration.

Member 62 includes a tissue engaging member, such as, for example, a blade 90 and member 64 includes a tissue engaging member, such as, for example, a blade 92. Blade 90 extends from member 68 and blade 92 extends from member 82. Blade 90 extends between an end 90a and an end 90b. Blade 90 includes an inner surface, such as, for example, a guide surface 94 and an outer surface 96 configured for engagement with tissue. Blade 92 extends between an end 92a and an end 92b. Blade 92 includes an inner surface, such as, for example, a guide surface 98 and an outer surface 100 configured for engagement with tissue. In some embodiments, all or only a portion of blade 90 and/or blade 92 may have various cross-section configurations, such as, for example, arcuate, cylindrical, oblong, rectangular, polygonal, undulating, irregular, uniform, non-uniform, consistent, variable, and/or U-shape.

Blades 90, 92 are independently, and/or relatively translatable and/or rotatable along axis X2 and axis A2. Blades 90, 92 are connected with members 62, 64 for movement, as described herein, to facilitate manipulation of tissue in and between one or a plurality of configurations and to space the tissue and define a configuration and dimension of a surgical opening, such as, for example, a surgical pathway, as described herein. In some embodiments, blade 90 and/or blade 92 includes an adjustable length such that a first portion of blade 90 and/or blade 92 translates relative to a second portion of blade 90 and/or blade 92.

Arms 14, 60 are independently and/or relatively translatable, as described herein. In some embodiments, members 62, 64 are configured for independent and/or relative rotation. Arm 14 extends from arm 60 in a co-axial and linear orientation. In some embodiments, arm 14 may be disposed in transverse orientations relative to arm 60, such as, for example, angular orientations such as acute or obtuse, and/or may be offset or staggered. In one embodiment, arm 14 includes an engagement portion, such as, example, a carriage comprising a rack 108 of a ratchet mechanism 106 for incremental and controlled independent and/or relative translation of arms 14, 60 to facilitate movement of blades 40, 42, 90, 92, which may include independent and/or relative translation of blades 40, 42 and blades 90, 92, as described herein. Rack 108 includes gear teeth 110 disposed in a linear configuration along arm 14 for engagement with arm 60 to facilitate incremental and controlled independent and/or relative translation of arms 14, 60 along axes X1, X2, as described herein.

Arms 14, 60 are engageable to facilitate incremental and controlled independent and/or relative translation of arms 14, 60 along axes X1, X2. In one embodiment, arm 60 includes an engagement portion, such as, example, pawls 112. Pawls 112 include gear teeth 114. Gear teeth 114 are configured for engagement with gear teeth 110 of rack 108 to facilitate incremental and controlled independent and/or relative translation of arms 14, 60 along axes X1, X2 to facilitate movement of blades 40, 42, 90, 92, as described herein. In some embodiments, arms 14, 60 include a rack and rotatable gear, such as, for example, a rack and pinion gear configuration.

In one embodiment, as shown in FIG. 3, blade 40 includes an actuating mechanism, such as, for example, a knob 111a configured to actuate selective and independent rotation of blade 40 about axis X1 and relative to member 22 between a first configuration and a second configuration to space tissue to define a configuration and dimension of a tissue opening and/or surgical pathway, as described herein. In one embodiment, blade 42 includes an actuating mechanism, such as, for example, a knob 111b configured to actuate selective and independent rotation of blade 42 about axis X1 and relative to member 32 between a first configuration and a second configuration to space tissue to define a configuration and dimension of a tissue opening and/or surgical pathway. In one embodiment, blade 90 includes an actuating mechanism, such as, for example, a knob 111c configured to actuate selective and independent rotation of blade 90 about axis X2 and member 68 between a first configuration and a second configuration to space tissue to define a configuration and dimension of a tissue opening and/or surgical pathway. In one embodiment, blade 92 includes an actuating mechanism, such as, for example, a knob 111d configured to actuate selective and independent rotation of blade 92 about axis X3 and member 82 between a first configuration and a second configuration to space tissue to define a configuration and dimension of a tissue opening and/or surgical pathway.

In some embodiments, blade 40 includes a guide surface 44 that defines an axis X3. Axis X3 is configured to provide a guide for a fastener to be connected with tissue, as described herein. In some embodiments, axis X3 can be aligned with an axis, such as, for example, a fastener axis F1 of a pilot hole for disposal of a bone screw. Guide surface 44 includes visual indicia 44a, such as, for example, radiomarkers for identification under x-ray, fluoroscopy, CT or other imaging techniques, colored markers, illuminated markers and/or projections. In some embodiments, the use of surgical navigation, microsurgical and image guided technologies may be employed to facilitate alignment of blade 40 with a pilot hole for disposal of a bone screw.

In some embodiments, blade 42 includes a guide surface 48 that defines an axis X4. Axis X4 is configured to provide a guide for a fastener to be connected with tissue, as described herein. In some embodiments, axis X4 can be aligned with an axis, such as, for example, a fastener axis F2 of a pilot hole for disposal of a bone screw. Guide surface 48 includes visual indicia 48a, such as, for example, radiomarkers for identification under x-ray, fluoroscopy, CT or other imaging techniques, colored markers, illuminated markers and/or projections. In some embodiments, the use of surgical navigation, microsurgical and image guided technologies may be employed to facilitate alignment of blade 42 with a pilot hole for disposal of a bone screw.

In some embodiments, blade 90 includes a guide surface 94 that defines an axis X5. Axis X5 is configured to provide a guide for a fastener to be connected with tissue, as described herein. In some embodiments, axis X5 can be aligned with an axis, such as, for example, a fastener axis F3 of a pilot hole for disposal of a bone screw. Guide surface 94 includes visual indicia 94a, such as, for example, radiomarkers for identification under x-ray, fluoroscopy, CT or other imaging techniques, colored markers, illuminated markers and/or projections. In some embodiments, the use of surgical navigation, microsurgical and image guided technologies may be employed to facilitate alignment of blade 90 with a pilot hole for disposal of a bone screw.

In some embodiments, blade 92 includes a guide surface 98 that defines an axis X6. Axis X6 is configured to provide a guide for a fastener to be connected with tissue, as described herein. In some embodiments, axis X6 can be aligned with an axis, such as, for example, a fastener axis F4 of a pilot hole for disposal of a bone screw. Guide surface 98 includes visual indicia 98a, such as, for example, radiomarkers for identification under x-ray, fluoroscopy, CT or other imaging techniques, colored markers, illuminated markers and/or projections. In some embodiments, the use of surgical navigation, microsurgical and image guided technologies may be employed to facilitate alignment of blade 92 with a pilot hole for disposal of a bone screw.

In some embodiments, one or more surgical tools or instruments I are disposed adjacent, connected, engaged and/or attached with one or more of blades 40, 42, 90, 92 and/or one or more of guide surfaces 44, 48, 94, 98. In some embodiments, surgical instrument I is engaged with guide surface 94 such that retractor 12 stabilizes surgical instrument I for delivering or introducing a bone fastener 120 with tissue adjacent a surgical site, for example, as shown in FIG. 5. In some embodiments, surgical instrument I can include a drill, awl, tap, driver, probe, sleeve and/or cannula and may be employed with a blade or guide surface to form a cavity or hole for an initial trajectory for bone fastener 120.

Surgical system 10 is employed, for example, with a minimally invasive procedure, including mini-open surgical techniques to deliver and introduce instrumentation and/or an implant, such as, for example, a bone fastener 120, at a surgical site within a body of a patient, which includes, for example, a spine having vertebrae V, as shown in FIG. 5.

In assembly, operation and use, surgical system 10, similar to the systems and methods described herein, is employed with a surgical procedure for treatment of a spinal disorder, such as those described herein, affecting a section of a spine of a patient. Spinal implant system 10 may also be employed with other surgical procedures. In some embodiments, surgical system 10 is employed to implant components, such as bone fasteners, rods, interbody devices and plates, with the body.

With the body disposed in a selected orientation, a medical practitioner makes and/or creates an incision in tissue, which includes soft tissue and/or muscle, to obtain access to a surgical site including vertebral levels V1, V2. In some embodiments, the tissue comprises cephalad portion CEP and caudad portion CAP disposed adjacent to the incision. The tissue comprising cephalad portion CEP and caudad portion CAP is manipulated in a cephalad-caudal orientation along a sag ittal plane to space the tissue adjacent to the incision. Manipulation of cephalad portion CEP and caudad portion CAP creates an access path to a surgical site including vertebrae V.

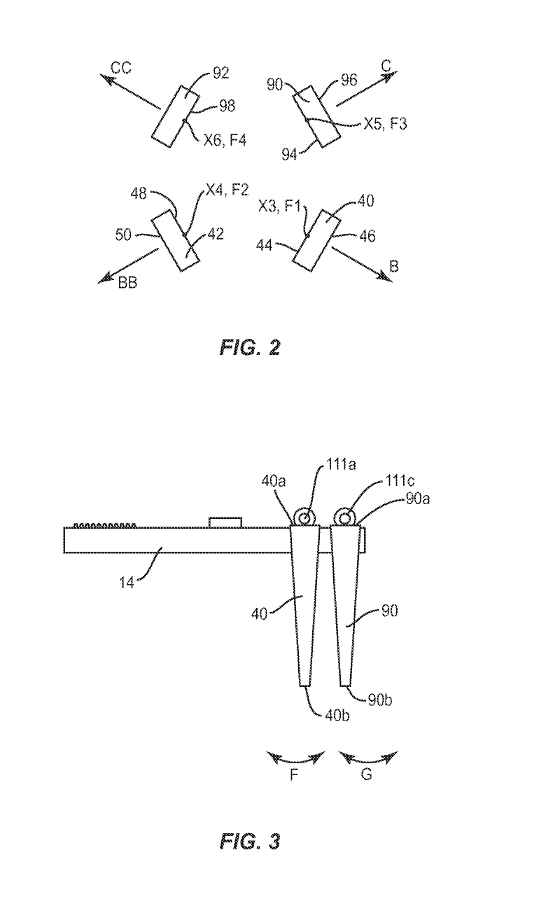

Retractor 12 is inserted through the incision and is disposed with the tissue to create a surgical pathway and/or opening to the surgical site. Blades 40, 42 engage and space tissue of caudad portion CAP adjacent to the incision. Blades 90, 92 engage and space tissue of cephalad portion CEP adjacent to the incision. Blades 40, 42, 90, 92 are independently and selectively movable, in the directions shown by arrows B, C, B, BB in FIG. 2, to space tissue portions CAP, CEP adjacent to the incision to define a configuration and dimension of a surgical pathway, which includes an opening S.

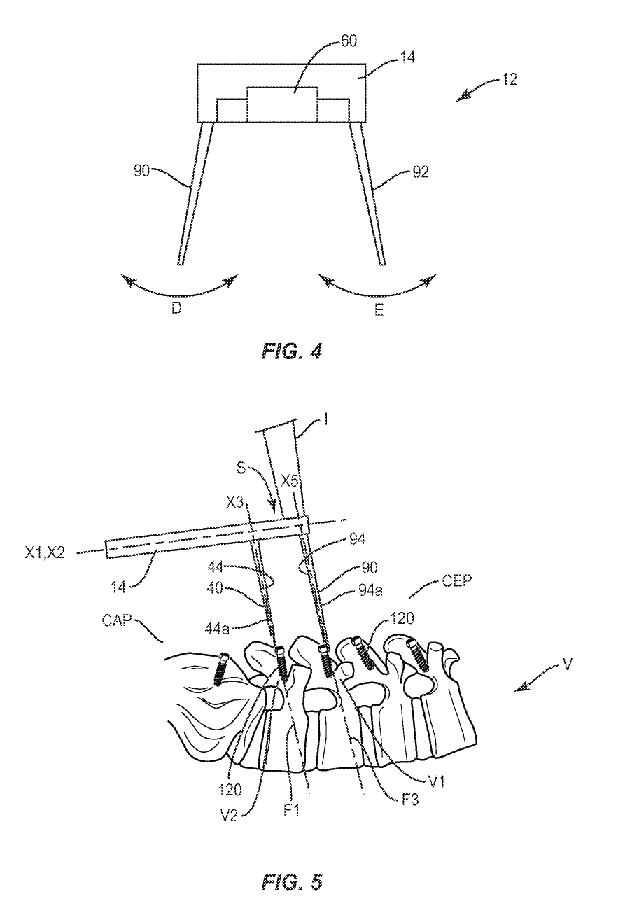

Arm 14 is translated along axes X1, X2 relative to arm 60 to independently translate and position blades 40, 42 relative to blades 90, 92. Member 16 is translated along axis A1 relative to member 18 to independently translate and position blade 40 relative to blade 42, and/or blades 40, 42 relative to blades 90, 92. Member 62 is translated along axis A2 relative to member 64 to independently translate and position blade 90 relative to blade 92, and/or blades 90, 92 relative to blades 40, 42. Knobs 111a, 111b, 111c and/or 111d, are independently actuated to independently rotate blades 40, 42, 90, 92, in the directions shown by arrows D, E, F, G in FIGS. 3 and 4. Blades 40, 42, 90, 92 are independently and/or relatively translated, rotated and positioned, as described herein, to manipulate tissue in and between one or a plurality of configurations and to space the tissue adjacent the incision and define a configuration and dimension of a surgical pathway, which includes opening S.

In some embodiments, blade 40, blade 42, blade 90 and/or blade 92 orient their respective guide surfaces to provide a guide for bone fasteners 120 to be aligned with pilot holes in vertebrae V and connected, for example, with vertebrae V1, V2, as described herein. In one embodiment, guide surface 44 of blade 40 is independently and/or relatively translated, rotated and positioned, as described herein, to manipulate tissue such that axis X3 is aligned with axis F1 of a pilot hole for disposal and fixation of a bone fastener 120 with vertebra V2, as shown in FIG. 5. In one embodiment, guide surface 94 of blade 90 is independently and/or relatively translated, rotated and positioned, as described herein, to manipulate tissue such that axis X5 is aligned with axis F3 of a pilot hole for disposal and fixation of a bone fastener 120 with vertebra V1.

Upon completion of a procedure, as described herein, the surgical instruments, assemblies and non-implanted components of surgical system 10 are removed and the incision(s) are closed. One or more of the components of surgical system 10 can be made of radiolucent materials such as polymers. Radiopaque markers may be included for identification under x-ray, fluoroscopy, CT or other imaging techniques. In some embodiments, the use of surgical navigation, microsurgical and image guided technologies, as described herein, may be employed to access, view and repair spinal deterioration or damage, with the aid of surgical system 10. In some embodiments, surgical system 10 may include implants and/or spinal constructs, which may include one or a plurality of plates, rods, connectors and/or bone fasteners for use with a single vertebral level or a plurality of vertebral levels.

It will be understood that various modifications and/or combinations may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first arm defining a longitudinal axis and including at least one tissue engaging member being movable along a transverse axis; and
a second arm being translatable relative to the first arm and including at least one tissue engaging member being movable along a transverse axis thereof,
the tissue engaging members being relatively movable between a first configuration and a second configuration to space tissue.

2. A surgical instrument as recited in claim 1, wherein the at least one tissue engaging member of the first arm is movable relative to the at least one tissue engaging member of the second arm.

3. A surgical instrument as recited in claim 1, wherein the transverse axis of the first arm is parallel to the transverse axis of the second arm.

4. A surgical instrument as recited in claim 1, wherein the transverse axis of the first arm is transverse to the transverse axis of the second arm.

5. A surgical instrument as recited in claim 1, wherein the second arm translates co-axially with the first arm.

6. A surgical instrument as recited in claim 1, wherein the at least one tissue engaging member of the first arm is rotatable relative to the first arm.

7. A surgical instrument as recited in claim 6, wherein the at least one tissue engaging member of the second arm is rotatable relative to the second arm.

8. A surgical instrument as recited in claim 1, wherein the tissue is disposed adjacent vertebrae and the at least one tissue engaging member of the first arm is rotatable relative to the first arm in a sagittal plane of the vertebrae.

9. A surgical instrument as recited in claim 1, wherein the tissue is disposed adjacent vertebrae and the at least one tissue engaging member of the first arm is rotatable relative to the first arm in a transverse plane of the vertebrae.

10. A surgical instrument as recited in claim 1, wherein the at least one tissue engaging member of the first arm includes a first blade and a second blade, the blades being independently movable.

11. A surgical instrument as recited in claim 10, wherein the at least one tissue engaging member of the second arm includes a first blade and a second blade, the blades of the first and second arms being independently movable.

12. A surgical instrument as recited in claim 1, wherein the at least one tissue engaging members of the first and second arms include a plurality of blades that are independently and selectively movable to space tissue and define a configuration and dimension of an opening therebetween.

13. A surgical instrument as recited in claim 1, wherein the at least one tissue engaging members each include a guide surface for a fastener axis.

14. A surgical instrument as recited in claim 13, wherein the guide surface includes visual indicia for alignment with the fastener axis.

15. A surgical instrument comprising:
a first arm defining a longitudinal axis and including a first blade and a second blade, the blades being translatable along a transverse axis and rotatable relative to the first arm; and
a second arm being translatable relative to the first arm, the second arm including a first blade and a second blade, the blades of the second arm being translatable along a transverse axis thereof and rotatable relative to the second arm,
the blades of the first and second arms being independently and selectively movable between a first configuration and a second configuration to space tissue.

16. A surgical instrument as recited in claim 15, wherein the tissue is disposed adjacent vertebrae and the blades of the first arm are rotatable relative to the first arm in a sagittal plane of the vertebrae.

17. A surgical instrument as recited in claim 15, wherein the tissue is disposed adjacent vertebrae and the blades of the first arm are rotatable relative to the first arm in a transverse plane of the vertebrae.

18. A surgical instrument as recited in claim 15, wherein the blades of the first and second arms each include a guide surface for a fastener axis, the guide surface being engageable with a surgical tool connected with a fastener.

19. A surgical instrument as recited in claim 18, wherein each of the guide surfaces includes visual indicia for alignment with the fastener axis.

20. A surgical system comprising:
a surgical instrument comprising a first arm defining a longitudinal axis and including at least one tissue engaging member being movable along a transverse axis, and a second arm being translatable relative to the first arm and including at least one tissue engaging member being movable along a transverse axis thereof, at least one of the tissue engaging members including a guide surface; and
at least one fastener defining a fastener axis,
wherein the tissue engaging members are relatively movable to space tissue and the guide surface includes visual indicia for alignment with the fastener axis.
